(19) United States
(12) Patent Application Publication
(10) Pub. No.: US 2008/0047559 A1
(43) Pub. Date: Feb. 28, 2008

(54) NASAL POSITIVE PRESSURE VENTILATION APPARATUS AND METHOD

(76) Inventor: ROMEO FIORI, Edgewater, NJ (US)

Correspondence Address: ROBERT S. SMITH, 1131-0 TOLLAND TURNPIKE, SUITE 306, MANCHESTER, CT, 06040, US

(21) Appl. No.: 11/466,429

(22) Filed: Aug. 22, 2006

(51) Int. Cl. A62B 7/10; A61M 15/08; A61M 16/00

(52) U.S. Cl. 128/206.11; 128/207.18

(57) ABSTRACT

A nasal positive pressure ventilation apparatus which includes a manifold and first and second nasal engagement knobs carried on the manifold, each of the nasal engagement knobs having a central passageway for gas being supplied to the respiratory system of an associated patient; the central passageway of each knob being disposed in fluid communication being with the manifold, each knob having at least a portion of the outer surface manufactured of a material that allows expansion, the first and second knobs having at least a portion of the outer surface thereof disposed over respective first and second chambers, a pump for forcing air into both the first and second chambers. The invention also includes the method for providing nasal positive pressure ventilation which includes providing a manifold and providing first and second nasal engagement knobs on the manifold having a central passageway for gas being supplied to the respiratory system of an associated patient; providing fluid communication between the central passageway of each knob with the manifold, providing each knob with at least a portion of the outer surface a material that allows expansion and is impervious to fluids, disposing at least a portion of the outer surface of the first and second knobs over respective first and second chambers, and providing a pump for forcing air into both the first and second chambers.

### FIELD OF THE INVENTION

The invention has particular application to methods and apparatus for noninvasive positive pressure ventilation to assist the respiratory system of a medical patient and has particular application to such systems that connect to the nasal passages of a patient. Thus, the present invention is intended for situations that do not involve the use of endotracheal tubes. Such apparatus and methods are employed, for example, in the treatment of sleep apnea and emphysema.

### BACKGROUND OF THE INVENTION

The prior art includes nasal cannula (also spelled canula used in a hospital or at home to deliver supplemental oxygen to a patient or person in need of extra oxygen. This typical system includes a plastic tube which fits around the head of a person and a set of two prongs which are placed in the nose or nares of the patient. These prongs are where the oxygen flows out of. To get oxygen through the nasal cannula it has to be hooked up to an oxygen tank, portable oxygen generator, or to a wall connection in a hospital via a flowmeter. The nasal cannula has a flow which ranges from 1 liters per minute to 6 liters per minute. There are also infant or neonatal nasal cannulas which use flows of less than a liter per minute and have smaller prongs. Typically, the gas flowing through a cannula has an oxygen content that ranges from 24% oxygen to 35% approximately.

There are three commonly used ways of delivering nasal positive pressure ventilation (NPPV):

Continuous positive airway pressure (CPAP), in which the machine delivers air at a constant positive pressure during inspiration and expiration

Volume-cycled, flow-limited, in which the machine delivers a set tidal volume each time the patient begins to take a breath

Pressure-limited, which in turn can be of three types:

### Pressure support, in which the machine delivers air at a set pressure during inspiration every time the patient starts to take a breath

### Pressure control, in which the machine automatically delivers a set number of breaths per minute at a set pressure

### Bilevel positive airway pressure (BiPAP), in which the machine delivers different pressures during inspiration and expiration.

Typically, plastic tubes connected to nasal cannula are used although in some cases, a full-face mask, a nasal mask, nasal “pillows,” or a mouthpiece may be utilized. The prior art includes U.S. Pat. No. 5,687,715 issued on Nov. 18, 1997 to Landis et al. which describes nasal delivery elements that are generally frustoconical in shape and are made of a soft, compliant material, e.g. silicone or polyurethane, to conform to the shape of the patient's nares (the passageways in the nose of the patient) and form a sealed therewith.

The prior art apparatus and method has not been wholly satisfactory. In some cases the mounting system used with conventional nasal cannula requires that the plastic tubing coupled to the nasal cannula be secured to the ears of the user to provide sufficient stability. The apparatus for securing the tubing may in some cases cause irritation and result in the source In some cases leakage at the nasal cannula may disturb the sleep of the user or others sleeping near the user.

### SUMMARY OF THE INVENTION

It is an object of the present invention to provide a method and apparatus that will minimize the need to mount the tubing of such systems on the ears of the user and thus minimize the occurrence of sores and irritation of the user's skin.

Another object of the present invention is to provide apparatus that will minimize leakage intermediate the nasal cannula and the and nares of the user's nose and thus will also eliminate the noise associated with such leakage.

Still another object invention is to provide an apparatus and method that will be simple to utilize.

Yet another object of the present invention is to provide an apparatus and method that will not compromise the safety of the user.

It has now been found that these and other objects of the present invention may be attained in a nasal positive pressure ventilation apparatus which includes a manifold; and first and second nasal engagement knobs carried on said manifold, each of said nasal engagement knobs having a central passageway for gas being supplied to the respiratory system of an associated patient; said central passageway of each knob being disposed in fluid communication with said manifold, each knob having at least a portion of the outer surface manufactured of a material that allows expansion, said first and second knobs having at least a portion of the outer surface thereof disposed over respective first and second chambers, a pump for forcing air into both said first and second chambers.

In some forms of the apparatus in accordance with the present invention the pump is a bulb having a check valve that allows air to enter the bulb and does not allow air to pass through the check valve from the interior of the bulb.

The manifold may be elongated and said knobs may be disposed on an elongated side. The bulb may be connected to the chamber by means of a tube having first and second axial sections joined by a connector. Some forms of the apparatus have an elongated manifold that has inlets at the axial extremities thereof.

Various embodiments of the apparatus in accordance with the present invention include a first outlet for passage of gases exhaled by the associated patient. Still other embodiments include a second outlet for passage of gases exhaled by the associated patient. The first and second outlets for passage of gases exhaled by the associated patient may be disposed in substantially coaxial relationship respectively with said first and second knobs.

The invention also includes the method for providing nasal positive pressure ventilation which includes providing a manifold and providing first and second nasal engagement knobs on said manifold having a central passageway for gas being supplied to the respiratory system of an associated patient; providing fluid communication between the central passageway of each knob with the manifold, providing each knob with at least a portion of the outer surface a material that allows expansion and is impervious to fluids, disposing at least a portion of the outer surface of the first and second knobs over respective first and second chambers, and providing a pump for forcing air into both said first and second chambers.

In some forms of the method in accordance with the present invention the step of providing a pump includes providing a bulb having a check valve that allows air to enter the bulb and does not allow air to pass through the check valve from the interior of the bulb.

The step of providing a manifold may include providing a manifold that is elongated with the knobs are disposed on an elongated side thereof and may include providing a connection from the bulb to the chamber by means of a tube having first and second axial sections joined by a connector.

Some forms of the method may include a step of providing a manifold that includes providing a manifold that has inlets at the axial extremities thereof. Other forms may include a step of providing a manifold that includes providing a manifold that includes a first outlet for passage of gases exhaled by the associated patient.

The step of providing a manifold may include providing a manifold that includes a second outlet for passage of gases exhaled by the associated patient.

In addition, the step of providing a manifold many include providing a manifold that has first and second outlets for passage of gases exhaled disposed in substantially coaxial relationship respectively with said first and second knobs.

### BRIEF DESCRIPTION OF THE DRAWING

The invention will be better understood by reference to the accompanying drawing in which:

FIG. 1 is a partially schematic view of a preferred form of the apparatus in accordance with one form of the invention that illustrates the outer cuffs of the nasal engagement knobs in the deflated position.

FIG. 2 is a partially schematic view illustrating in greater detail a portion of the apparatus shown in FIG. 1 and which additionally illustrates the outer cuffs of the nasal engagement knobs in the inflated position.

### DESCRIPTION OF THE PREFERRED EMBODIMENTS

The following describes a preferred embodiment of the present invention soon will for use in a continuous positive airway pressure system in which the delivers air at a constant pressure during both inspiration and expiration. Those skilled in the art will recognize that the apparatus and method has application to other ventilation apparatus, including but not limited to, volume cycled and pressure-limited systems.

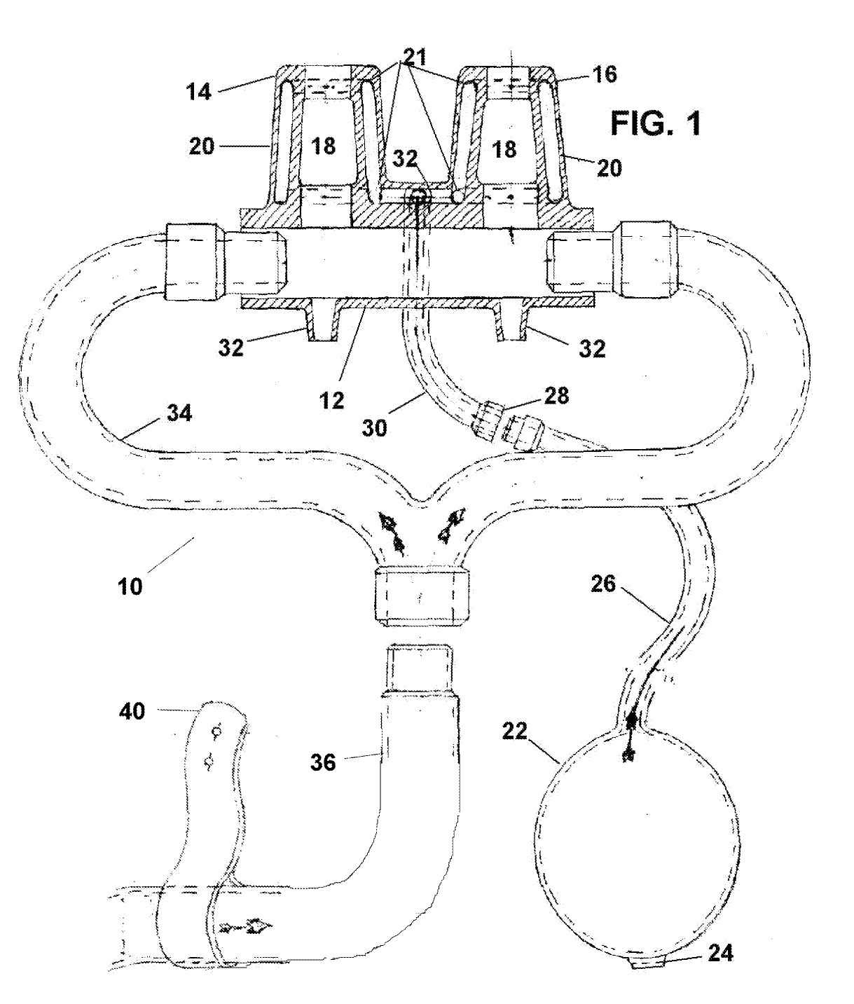

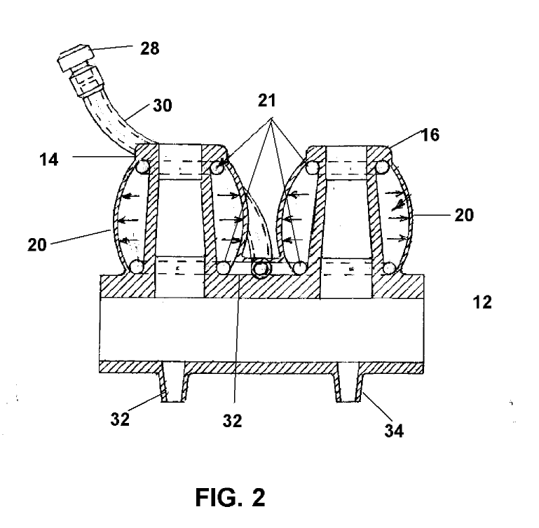

Referring now to FIGS. 1 and 2 there is shown one form of the apparatus 10 in accordance with the present invention. The apparatus 10 includes a manifold 12 that includes first and second nasal engagement frustoconical knobs 14, 16. The knobs 14, 16 are manufactured of silicone or polyurethane or other soft material that will provide a good seal with respect to the nares of the users nostrils. Some embodiments may also use latex rubber for those patients that are not allergic to the material. Each of the knobs 14, 16 has a central bore defined by a sleeve. The outer surface of each of the knobs 14, 16 is a cuff 20. Typically, the structure of the sleeve defining the passageway 18 will be more robust than the structure of the cuff 20. This may be achieved by the use of different materials or the use of materials having a different thickness.

The difference in robustness of the sleeve defining the passageway 18 and the outer cuff 20 provides, with the structure to be described hereafter, the ability to inflate the cuffs 2 to provide a positive seal with the nares of the users nose with no discomfort to the user. In the preferred embodiment the inflation of the cuffs 20 is achieved by a rubber bulb 22 having an inlet 24 equipped with a check valve that allows air to only enter the interior of the ball. Thus, compression of the bulb 22, typically made of rubber, forces air out of the ball 22 into tube 26, through connector 28, through tube 30, into the elongated chamber 32 (within the manifold 12) and then into the space intermediate the sleeve defining the passageway 18 and the cuff 20. Passageways 21 provide fluid communication to the space intermediate the sleeve defining a passageway 18 and the cuff 20. A strap 40 is used to provide support for the lower parts of the tube 36. Typically the strap 40 is attached to a button on the clothing of the user.

In accordance with the customary practice in continuous positive airway pressure (CPAP) systems, in which air is delivered at a constant positive pressure during both inspiration and expiration the manifold 12 is provided with outlets 32, 32. The outlets 32, 32 in the preferred embodiment are respectively disposed in substantially coaxial relationship with the knobs 14, 16.

Oxygen rich gases are supplied to the manifold 12 by means of a yoke 34 having respective tubular portions coupled to opposed axial extremities of the elongated manifold 12. The yoke 34 is coupled to a hose 36 which is in turn coupled to an associated source of the oxygen rich gas (not shown).

The invention includes both the apparatus described above as well as the method of providing a seal with the nares of a patient's nose utilizing inflatable cuff. It will be understood that the patient will connect the knobs 14, 16 to the nares of his or her nose by inserting the knobs and then squeezing the bulb 22 to pump air into the chambers between the cuff 20 and a sleeve that defines the passageway 18. This connection of the coupling 28 allows the cuffs 22 to deflate and facilitates removal of the apparatus. The method, as well as the use of the apparatus, may include squeezing the tube 30 after the cuffs 20 have been inflated while simultaneously positioning a cap (not shown) on the end of the tube 30 to trap the inflation air in the cuff 20. Other methods and apparatus in accordance with the invention may include a check valve (not shown) to allow air flow in to inflate the cuff 20. An exhaust valve (not shown) may also be provided to allow the user to selectively exhaust the air that inflates the cuff.

It will thus be seen at the method and apparatus in accordance with present invention deletes the requirement for many of the straps and other structure used by the prior art apparatus. This eliminates much of the chafing and irritation caused by the prior apparatus. In addition the superior seal that is possible with the present apparatus and method ensures that leakage will not produce noise that will be annoying or disturb the sleep of the patient or those around the patient.

Although the description above contains many specifics, these should not be construed as limiting the scope of the invention, but as merely providing illustrations of some of the presently preferred embodiments of this invention. Thus, the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. Nasal positive pressure ventilation apparatus which comprises:
a manifold; and
first and second nasal engagement knobs carried on said manifold, each of said nasal engagement knobs having a central passageway for gas being supplied to the respiratory system of an associated patient; said central passageway of each knob being disposed in fluid communication being with said manifold, each knob having at least a portion of the outer surface manufactured of a material that allows expansion, said first and second knobs having at least a portion of the outer surface thereof disposed over respective first and second chambers, a pump for forcing air into both said first and second chambers.

2. The apparatus as described in claim 1 wherein said pump is a bulb having a check valve that allows air to enter the bulb and does not allow air to pass through the check valve from the interior of the bulb.

3. The apparatus as described in claim 1 wherein said manifold is elongated and said knobs are disposed on an elongated side.

4. The apparatus as described in claim 2 wherein said bulb is connected to said chamber by means of a tube having first and second axial sections joined by a connector.

5. The apparatus as described in claim 4 wherein said manifold has inlets at the axial extremities thereof.

6. The apparatus as described in claim 5 wherein said manifold includes a first outlet for passage of gases exhaled by the associated patient.

7. The apparatus as described in claim 6 wherein said manifold includes a second outlet for passage of gases exhaled by the associated patient.

8. The apparatus as described in claim 7 wherein said first and second outlets for passage of gases exhaled by the associated patient are disposed in substantially coaxial relationship respectively with said first and second knobs.

9. A method for providing nasal positive pressure ventilation which comprises:
providing a manifold;
providing first and second nasal engagement knobs on said manifold having a central passageway for gas being supplied to the respiratory system of an associated patient;
providing fluid communication between the central passageway of each knob with the manifold, providing each knob with at least a portion of the outer surface a material that allows expansion and is impervious to fluids, disposing at least a portion of the outer surface of the first and second knobs over respective first and second chambers, and providing a pump for forcing air into both said first and second chambers.

10. The method as described in claim 9 wherein said step of providing a pump includes providing a bulb having a check valve that allows air to enter the bulb and does not allow air to pass through the check valve from the interior of the bulb.

11. The method as described in claims 9 wherein said step of providing a manifold includes providing a manifold that is elongated with the knobs are disposed on an elongated side thereof.

12. The method as described in claim 10 wherein the step of providing a pump includes providing a connection from the bulb to the chamber by means of a tube having first and second axial sections joined by a connector.

13. The method as described in claim 12 wherein the step of providing a manifold includes providing a manifold that has inlets at the axial extremities thereof.

14. The method as described in claim 13 wherein the step of providing a manifold includes providing a manifold that includes a first outlet for passage of gases exhaled by the associated patient.

15. The method as described in claim 14 wherein the step of providing a manifold includes providing a manifold that includes a second outlet for passage of gases exhaled by the associated patient.

16. The method as described in claim 15 wherein the step of providing a manifold includes providing a manifold that has first and second outlets for passage of gases exhaled disposed in substantially coaxial relationship respectively with said first and second knobs.
